(19) United States
(12) Patent Application Publication
(10) Pub. No.: US 2006/0200048 A1
(43) Pub. Date: Sep. 7, 2006

(54) Removable sheath for device protection

(75) Inventors: Joseph G. Furst, Lyndhurst, OH (US); Ravish Sachar, Raleigh, NC (US)

Correspondence Address: Fay, Sharpe, Fagan,;Minnich & McKee, LLP, 7th Floor, 1100 Superior Avenue, Cleveland, OH, 44114-2579, US

(73) Assignee: ICON Medical Corp.

(21) Appl. No.: 11/349,296

(22) Filed: Feb. 7, 2006

Related U.S. Application Data
(60) Provisional application No. 60/658,404, filed on Mar. 3, 2005.

(51) Int. Cl. A61M 25/00

(52) U.S. Cl. 600/585

(57) ABSTRACT

A medical device designed to be used with a guide catheter. The medical device has a body and cross-sectional shape and size that enables the guide catheter to be at least partially fed through a guide catheter. The body of the medical device has a longitudinal length that is at least about 10% of a longitudinal length of the guide catheter. The body of the medical device is at least partially formed of a flexible material.

The present invention claims priority on U.S. Provisional Patent Application Ser. No. 60/658,404 filed Mar. 3, 2005, which is incorporated herein by reference.

The invention relates generally to medical devices, and more particularly to a device that can be used with an implant and/or balloon or catheter for use in body passageways, and still even more particularly to a sheath-like device for use in body passageways to assist in the treatment of stenoses in the vascular system.

### BACKGROUND OF THE INVENTION

Heart disease is one of, if not, the highest occurring disease affecting humans. Currently millions of people world wide are affected by some form of heat disease. Old age, dietary habits and primary genetics can also lead to a common disease, atherosclerosis. Atherosclerotic plaques and blockages consist of lipids, fibroblasts and fibrin that proliferate and cause obstruction of a vessel. As the obstruction grows, the blood flow diminishes and reaches a level that is insufficient to meet the biological needs of one or more organs. The end result is defined as ischemia. Heart disease commonly occurs when an artery and/or vein is partially or fully obstructed with various biological entities such as, but not limited to, platelets, plaque, cholesterol, calcium, etc. Not only is the heart adversely affected by the partial or full obstruction of a artery and/or vien, but other vasculature throughout the mammalian anatomy is also adversely affected.

To correct many types of heart disease, a surgical or interventional procedure is commonly preformed. A surgical procedure can include bypass surgery to the heart and/or the use of an artificial graft surgically sown on an artery and/or vein to restore blood flow. Another type of surgical procedure that can be used involves the use of a a stent, an angioplasty balloon, etc. This type of surgical procedure in preferred in many instances since it is a less invasive means for treating heart disease. This interventional procedure commonly involves the placement of a stent or angioplasty balloon into the area of disease, or the use of a cutting or removal device directed into the area of disease to open a previously clogged conduit, thus restoring fluid flow (e.g., blood flow).

The one of the primary purposes of a stent is to open a blocked or partially blocked body passageway. When a stent is used in a blood vessel, the stent is used to open the occluded vessel to achieve improved blood flow which is necessary to provide the anatomical function of an organ. The procedure of opening a blocked or partially blocked body passageway commonly includes one or more stents in combination with other medical devices such as, but not limited to, an introducer sheath, a guiding catheter, a guide wire, an angioplasty balloon, etc.

During the insertion of the stent into a body passageway, several problems can occur such as a) the stent becoming dislodged from the angioplasty balloon, b) the angioplasty balloon and/or stent being damaged, c) the guide wire being damaged, d) the angioplasty balloon not properly disengaging from the stent, and/or e) the stent not being able to pass minor artery deposits so that the stent can be positioned in a major blockage area. In the past when one or more of these problems occurred, the complete guide wire, angioplasty balloon and/or stent had to be fully withdrawn from the guide cathether, and the complete re-positioning procedure for the stent, wire and/or angioplasty balloon was then repeated. In many situation, the guide cathether had to be partially or fully retracted to allow blood flow through a treated region. Such a process can result in increased surgical times and costs, and may increase the health risks to the patient.

In view of the present state of medical device technology, there is a need and demand for a medical device and/or medical procedure that can be used to overcome the past problems associated with the delivery of a stent and/or angioplasty balloon into a body passageway.

### SUMMARY OF THE INVENTION

The invention relates to a medical device that can be used with a treatment device (e.g., stent, balloon, guide wire, etc.) in body passageways. As defined herein, the term “body passageway” is defined to be any passageway or cavity in a living organism (e.g., bile duct, bronchiole tubes, nasal cavity, blood vessels, heart, esophagus, trachea, stomach, fallopian tube, uterus, ureter, urethra, the intestines, lymphatic vessels, nasal passageways, eustachian tube, acoustic meatus, etc.). The techniques employed to deliver the medical device to a treatment area include, but are not limited to, angioplasty, vascular anastomoses, transplantation, implantation, subcutaneous introduction, minimally invasive surgical procedures, and any combinations thereof. As can be appreciated, other or additional techniques may be used. For vascular applications, the term “body passageway” primarily refers to blood vessels and chambers in the heart. In one non-limiting embodiment of the invention, the medical device is designed as a sheath-like device for use in body passageways. In another and/or alternative non-limiting embodiment of the invention, the medical device is designed as a sheath-like device for use in body passageways to assist in the treatment of stenoses in the vascular system. During an interventional procedure, many techniques can be utilized to attempt to treat a blocked or partially blocked passageway. The present invention is directed to a medical procedure and medical device that assists in reducing or preventing the failure of these various interventional procedures to thereby improve the success rate of such procedures.

In one non-limiting aspect of the invention, there is provided a medical device that is at least partially in the form of a generally tubular member. As can be appreciated, the medical device can have other cross-sectional shapes. The medical device can have a generally uniform cross-sectional shape and size along the longitudinal length of the medical device. As can be appreciated, the medical device can have cross-sectional shape and/or have a cross-sectional size that varies along the longitudinal length of the medical device. The medical device is at least partially formed of one or more flexible materials. Such flexible materials can include, but are not limited to, man-made plastics and/or polymers, natural polymers (e.g., rubber, etc.), plant fibers, metals, fiber reinforced materials (e.g., fiberglass, carbon fiber materials, etc.), etc. In one non-limiting embodiment of the invention, a majority of the medical device is formed of one or more flexible materials. In one non-limiting aspect of this embodiment, over 60% of the medical device is formed of one or more flexible materials. In another non-limiting aspect of this embodiment, over 80% of the medical device is formed of one or more flexible materials. In still another non-limiting aspect of this embodiment, over 90% of the medical device is formed of one or more flexible materials.

In another and/or alternative non-limiting aspect of the invention, there is provided a medical device that includes one or more slits along a longitudinal axis of the medical device. At least one of the one or more slits can enable the cross-sectional size or area of one or more regions of the medical device to increase when one or more medical implements are moved partially or fully through the medical device. The one or more slits typically penetrate the body of the medical device; however, this is not required. In one non-limiting embodiment of the invention, the one or more slits are generally straight; however, this is not required. In another and/or alternative non-limiting embodiment of the invention, at least one slit extends at least partially along a the longitudinal length of the medical device. In one non-limiting aspect of this embodiment, at least one slit extends a majority of the longitudinal length of the medical device. In another non-limiting aspect of this embodiment, at least one slit extends at least about 60% of the longitudinal length of the medical device. In still another non-limiting aspect of this embodiment, at least one slit extends at least about 80% of the longitudinal length of the medical device. In yet another non-limiting aspect of this embodiment, at least one slit extends at least about 95% of the longitudinal length of the medical device. In still yet another non-limiting aspect of this embodiment, at least one slit extends 100% of the longitudinal length of the medical device.

In still another and/or alternative non-limiting aspect of the invention, there is provided a medical device that includes one or more markers to facilitate in the location and/or guiding of the medical device in a body passageway. The marker material is typically designed to be visible to electromagnetic waves (e.g., x-rays, microwaves, visible light, inferred waves, ultraviolet waves, etc.); sound waves (e.g., ultrasound waves, etc.); magnetic waves (e.g., MRI, etc.); and/or other types of electromagnetic waves (e.g., microwaves, visible light, inferred waves, ultraviolet waves, etc.). In one non-limiting embodiment, the marker material is visible to x-rays (i.e., radiopaque). The marker material can form all or a portion of the medical device and/or be coated on one or more portions (flaring portion and/or body portion; at ends of medical device; at or near transition of body portion and flaring section; etc.) of the medical device. The location of the marker material can be on one or multiple locations on the medical device. The size of the one or more regions that include the marker material can be the same or different. The marker material can be spaced at defined distances from one another so as to form ruler like markings on the medical device to facilitate in the positioning of the medical device in a body passageway. In one non-limiting embodiment of the invention, at least one marker is located on at least one distal end of the medical device; however, it can be appreciated that the one or more markers can be located in other or additional location on the medical device. In another and/or alternative non-limiting embodiment of the invention, the marker material can be a rigid or flexible material. In still another and/or alternative non-limiting embodiment of the invention, the marker material can be a biostable or biodegradable material. When the marker material is a rigid material, the marker material can be formed of a metal material (e.g., metal band, metal plating, etc.); however, other or additional materials can be used. The metal material can be secured in a variety of ways such as, but not limited to, crimping, adhesive, melting, etc. When the marker material is a flexible material, the marker material can be formed of one or more polymers that are marker materials in-of-themselves and/or include one or more metal powders and/or metal compounds. In one non-limiting aspect of this embodiment, the flexible marker material includes one or more metal powders in combinations with parylene, PLGA, POE, PGA, PLLA, PAA, PEG, chitosan and/or derivatives of one or more of these polymers. In another and/or alternative non-limiting aspect of this embodiment, the flexible marker material includes one or more metals and/or metal powders of aluminum, barium, bismuth, cobalt, copper, chromium, gold, iron, stainless steel, titanium, vanadium, nickel, zirconium, niobium, lead, molybdenum, platinum, yttrium, calcium, rare earth metals, rhenium, zinc, silver, depleted radioactive elements, tantalum and/or tungsten; and/or compounds thereof. In yet another and/or alternative non-limiting embodiment of the invention, the marker material can be coated with a polymer protective material; however, this is not required. When the marker material is coated with a polymer protective material, the polymer coating can be used to 1) at least partially insulate the marker material from body fluids, 2) facilitate in retaining the marker material on the medical device, 3) at least partially shielding the marker material from damage during a medical procedure and/or 4) provide a desired surface profile on the medical device. As can be appreciated, the polymer coating can have other or additional uses. The polymer protective coating can be a biostable polymer or a biodegradable polymer (e.g., degrades and/or is absorbed). The coating thickness of the protective coating polymer material, when used, is typically less than about 300 microns; however, other thickness can be used. In one non-limiting embodiment, the protective coating materials include parylene, PLGA, POE, PGA, PLLA, PAA, PEG, chitosan and/or derivatives of one or more of these polymers.

In yet another and/or alternative non-limiting aspect of the invention, there is provided a medical device that has an outer diameter or cross-sectional area that is generally less than the inner diameter or cross-sectional area of a guide catheter so that the medical device can be at least partially inserted or threaded through the guide catheter. Generally, the medical device has a cross-sectional area that is at least about 1% less than the cross-section area of the inner passageway of the guide catheter. Typically, the cross-sectional area of the medical device is about 5-50% less than the cross-section area of the inner passageway of the guide catheter; however, the medical device can have other sizes.

In still yet another and/or alternative non-limiting aspect of the invention, there is provided a medical device that includes an internal passageway. In one non-limiting embodiment of the invention, the internal passageway extends a majority of the longitudinal length of the medical device. In another non-limiting embodiment of the invention, the internal passageway extends at least about 60% of the longitudinal length of the medical device. In still another non-limiting embodiment of the invention, the internal passageway extends at least about 80% of the longitudinal length of the medical device. In still another non-limiting embodiment of the invention, the internal passageway extends at least about 95% of the longitudinal length of the medical device. In yet another non-limiting embodiment of the invention, the internal passageway extends 100% of the longitudinal length of the medical device. In another and/or alternative non-limiting embodiment of the invention, the internal passageway can have a uniform or varied diameter or cross-sectional area along the longitudinal length of internal passageway. In still another and/or alternative non-limiting embodiment of the invention, at least a portion of the diameter or cross-sectional area of the internal passageway of the medical device is greater than or equal to the outer diameter or cross-sectional area of the treatment device (e.g., stent, angioplasty balloon, etc.) so that the treatment device can be at least partially inserted or threaded through the internal passageway and/or at least partially moved within the internal passageway. In one non-limiting aspect of this embodiment, the diameter or cross-sectional area of at least a portion of the internal passageway is greater than or equal to the outer diameter or cross-sectional area of the treatment device. In another non-limiting aspect of this embodiment, the diameter or cross-sectional area of a majority of the internal passageway is greater than or equal to the outer diameter or cross-sectional area of the treatment device. In yet another and/or alternative non-limiting embodiment of the invention, the size and cross-sectional shape of the medical device and the internal passageway of the medical device in combination with the one or more slits along at least a portion of the longitudinal axis of the medical device enables the medical device to be 1) inserted at least partially through a guide catheter, if such a guide catheter is used, 2) inserted to a treatment area wherein one or more treatment devices are located in a body passageway, and 3) at least partially inserted about the one or more treatment devices that are located in a guide catheter and/or in the body passageway. The size and cross-sectional shape of the medical device and the internal passageway of the medical device in combination with the one or more slits along at least a portion of the longitudinal axis of the medical device enables one or more of the treatment devices to be at least partially moved through the internal passageway of the medical device.

In a further and/or alternative non-limiting aspect of the invention, there is provided a medical device that can be easily and/or conveniently moved and/or positioned in a body passageway and/or the guide catheter. In one non-limiting embodiment of the invention, there is provided a hub located at the proximal end of the medical device. In one non-limiting aspect of this embodiment, a portion of the hub can include a receiving cavity. In one non-limiting design, the cavity includes a slit or other arrangement to enable the hub to be connected to and/or engage the medical device, thereby enabling an end of the medical device to be permanently/releasably secured to the cavity. In another and/or alternative non-limiting aspect of this embodiment, the hub can include one or more gripping elements; however, this is not required. In still another and/or alternative non-limiting aspect of this embodiment, the hub can be designed to enable a user to better and more easily manipulate the medical device in the guide catheter and/or body passageway.

In still a further and/or alternative non-limiting aspect of the invention, there is provided a medical device that has a longitudinal length that is at least about 50% the length of the guide catheter. In one non-limiting embodiment of the invention, the medical device has a longitudinal length that is about 100-150% the length of the guide catheter. In another non-limiting embodiment of the invention, the medical device has a longitudinal length that is at least about 200% the length of the guide catheter. The longitudinal length of the medical device is selected to enable the medical device to perform its intended function. Various longitudinal lengths of the medical device can be selected for different medical treatments. When a treatment device is located in the guide catheter, the longitudinal length of the medical device can be selected to enable the medical device to be inserted into the guide catheter and to the location of the treatment device. When the treatment device is positioned in a body passageway, the longitudinal length of the medical device can be selected to enable the medical device to be inserted through the full length of the guide catheter and the distance from the end of the guide catheter to a location at least closely adjacent to the treatment device. In this particular design, the medical device has a longitudinal length that is typically greater than the guide catheter. Guide catheters that are commonly used for treatment of humans can a length of about 10-150 cm; however, other lengths can be used. The inner diameter size of the catheter is typically about 0.04-0.09 inch; however other sizes can be used. It is not uncommon for a treatment device to be positioned about 0.1-18 inches from the end of the guide catheter. As such, the medical device can be slightly or substantially longer than the guide catheter and have an outer diameter or cross-section area that is small enough to engage the medical device to be moved within the guide catheter.

In yet a further and/or alternative non-limiting aspect of the invention, there is provided a medical device that includes one or more reinforcing, stiffening and/or strengthening components. One or more of these components can be attached to and/or incorporated with the flexible material forming the medical device in one or more regions of the medical device. One or more of these components can be used to provide integrity to the medical device. One or more of these components can be uniformly positioned on the medical device or be used in certain regions of the medical device. One or more of these components can include, but are not limited to, metals, carbon fibers and/or other reinforming fibers, fiberglass, plastics, polymers, etc.

In still yet a further and/or alternative non-limiting aspect of the invention, there is provided a medical device that is partially or fully coated with a material that facilitates in the movement of the medical device in a guide catheter and/or body passageway, and/or facilitates in the movement of one or more treatment deices in the internal passageway of the medical device. The coating material can also or alternatively be used to reduce or prevent damage to a body passageway as the medical device is moved in the body passageway; however, this is not required. The coating material can be used to reduce the coefficient of friction on at least a portion of the outer surface of the medical device and/or inner surface of the internal passageway. The coating material can include, but is not limited to, a plastic, polymer, Teflon, silicone, etc.

In another and/or alternative non-limiting aspect of the invention, there is provided a medical device that includes one or more gripping surfaces to facilitate in the handling and/or manipulation of the medical device. In one non-limiting embodiment of the invention, the body of the medical device includes a gripping surface positioned at least one proximal end of the medical device. As can be appreciated, the medical device can include one or more gripping surfaces in other or additional regions of the medical device. The gripping surface can be formed from a coating material (e.g., rough polymer coating, rubber coating, etc.) and/or be formed in the material of the medical device (e.g., surface ribs, rough surface, etc.).

In still another and/or alternative non-limiting aspect of the present invention, a) one or more portions of the medical device can include, contain and/or be coated with one or more biological agents to facilitate in the success of the medical device and/or treated area, and/or b) the internal passageway of the medical device can be used as a conduit to direct one or more biological agents at or near a treatment area to facilitate in the success of the medical device and/or treated area. The term “biological agent” includes, but is not limited to, a substance, drug or otherwise formulated and/or designed to prevent, inhibit and/or treat one or more biological problems, and/or to promote the healing in a treated area. Non-limiting examples of biological problems that can be addressed by one or more biological agents include, but are not limited to, viral, fungus and/or bacteria infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; and/or the like. Non-limiting examples of biological agents that can be used include, but are not limited to, 5-Fluorouracil and/or derivatives thereof; 5-Phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; agents that modulate intracellular Ca₂₊ transport such as L-type (e.g., diltiazem, nifedipine, verapamil, etc.) or T-type Ca₂₊ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; anti-biotics; anti-coagulant compounds and/or derivatives thereof; anti-fibrosis compounds and/or derivatives thereof; anti-fungal compounds and/or derivatives thereof; anti-inflammatory compounds and/or derivatives thereof; Anti-Invasive Factor and/or derivatives thereof; anti-metabolite compounds and/or derivatives thereof (e.g., staurosporin, trichothecenes, and modified diphtheria and ricin toxins, Pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative and/or derivatives thereof; anti-thrombogenic agents and/or derivatives thereof; argatroban and/or derivatives thereof; ap-1 inhibitors and/or derivatives thereof (e.g., for tyrosine kinase, protein kinase C, myosin light chain kinase, Ca₂+/calmodulin kinase II, casein kinase II, etc.); aspirin and/or derivatives thereof; azathioprine and/or derivatives thereof; β-Estradiol and/or derivatives thereof; β-1-anticollagenase and/or derivatives thereof; calcium channel blockers and/or derivatives thereof; calmodulin antagonists and/or derivatives thereof (e.g., H₇, etc.); CAPTOPRIL and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; CILAZAPRIL and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin O, cytochalasin P, cytochalasin Q, cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof; endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP₃ receptor and/or derivatives thereof; inhibitors of the sodium/hydrogen antiporter and/or derivatives thereof (e.g., amiloride and derivatives thereof, etc.); insulin and/or derivatives thereof; Interferon alpha 2 Macroglobulin and/or derivatives thereof; ketoconazole and/or derivatives thereof; Lepirudin and/or derivatives thereof; LISINOPRIL and/or derivatives thereof; LOVASTATIN and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; Plasminogen Activator Inhibitor-1 and/or derivatives thereof; Plasminogen Activator Inhibitor-2 and/or derivatives thereof; Platelet Factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, I-123, I-125, I-131, Re-186, Re-188, Au-198, Au-199, Pb-203, At-211, Pb-212, Bi-212, H₃P³²O₄, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; Sulphated Polysaccharide Peptidoglycan Complex and/or derivatives thereof; T_{H1} and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; Tissue Inhibitor of Metalloproteinase-1 and/or derivatives thereof; Tissue Inhibitor of Metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof. As can be appreciated, the biological agent can include one or more derivatives of the above listed compounds and/or other compounds. The type and/or amount of biological agent can vary. When two or more biological agents are used, the amount of two or more biological agents can be the same or different. The type and/or amount of biological agent is generally selected for the treatment of one or more medical treatments. The amount of two of more biological agents can be the same or different. The one or more biological agents can be coated on and/or impregnated in the medical device by a variety of mechanisms such as, but not limited to, spraying (e.g., atomizing spray techniques, etc.), dip coating, roll coating, sonication, brushing, plasma deposition, depositing by vapor deposition.

In a further and/or alternative non-limiting aspect of the present invention, the one or more biological agents on and/or in the medical device, when used on the medical device, can be released in a controlled manner so the area in question to be treated is provided with the desired dosage of biological agent over a sustained period of time. As can be appreciated, controlled release of one or more biological agents on the medical device is not always required and/or desirable. As such, one or more of the biological agents on and/or in the medical device can be uncontrollably released from the medical device during and/or after insertion of the medical device in the treatment area. It can also be appreciated that one or more biological agents on and/or in the medical device can be controllably released from the medical device and one or more biological agents on and/or in the medical device can be uncontrollably released from the medical device. It can also be appreciated that one or more biological agents on and/or in one region of the medical device can be controllably released from the medical device and one or more biological agents on and/or in the medical device can be uncontrollably released from another region on the medical device. As such, the medical device can be designed such that 1) all the biological agent on and/or in the medical device is controllably released, 2) some of the biological agent on and/or in the medical device is controllably released and some of the biological agent on the medical device is non-controllably released, or 3) none of the biological agent on and/or in the medical device is controllably released. The medical device can also be designed such that the rate of release of the one or more biological agents from the medical device is the same or different. The medical device can also be designed such that the rate of release of the one or more biological agents from one or more regions on the medical device is the same or different. Non-limiting arrangements that can be used to control the release of one or more biological agent from the medical device include a) at least partially coat one or more biological agents with one or more polymers, b) at least partially incorporate and/or at least partially encapsulate one or more biological agents into and/or with one or more polymers, c) insert one or more biological agents in pores, passageway, cavities, etc. in the medical device and at least partially coat or cover such pores, passageway, cavities, etc. with one or more polymers, and/or incorporate one or more biological agents in the one or more polymers that at least partially form the medical device. As can be appreciated, other or additional arrangements can be used to control the release of one or more biological agent from the medical device. The one or more polymers used to at least partially control the release of one or more biological agent from the medical device can be porous or non-porous. The one or more biological agents can be inserted into and/or applied to one or more surface structures and/or micro-structures on the medical device, and/or be used to at least partially form one or more surface structures and/or micro-structures on the medical device. As such, the one or more biological agents on the medical device can be 1) coated on one or more surface regions of the medical device, 2) inserted and/or impregnated in one or more surface structures and/or micro-structures, etc. of the medical device, and/or 3) form at least a portion or be included in at least a portion of the structure of the medical device. When the one or more biological agents are coated on the medical device, the one or more biological agents can, but is not required to, 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials. As can be appreciated, many other coating arrangements can be additionally or alternatively used. When the one or more biological agents are inserted and/or impregnated in one or more portions of the medical device, one or more surface structure and/or micro-structures of the medical device, and/or one or more surface structures and/or micro-structures of the medical device, 1) one or more other polymers can be applied at least partially over the one or more surface structure and/or micro-structures, surface structures and/or micro-structures of the medical device, 2) one or more polymers can be combined with one or more biological agents, and/or 3) one or more polymers can be coated over or more portions of the body of the medical device; however, this is not required. As such, the one or more biological agents can be 1) embedded in the structure of the medical device; 2) positioned in one or more surface structure and/or micro-structures of the medical device; 3) encapsulated between two polymer coatings; 4) encapsulated between the base structure and a polymer coating; 5) mixed in the base structure of the medical device that includes at least one polymer coating; or 6) one or more combinations of 1, 2, 3, 4 and/or 5. In addition or alternatively, the one or more coatings of the one or more polymers on the medical device can include 1) one or more coatings of non-porous polymers; 2) one or more coatings of a combination of one or more porous polymers and one or more non-porous polymers; 3) one or more coating of porous polymer, or 4) one or more combinations of options 1, 2, and 3. As can be appreciated different biological agents can be located in and/or between different polymer coating layers and/or on and/or the structure of the medical device. As can also be appreciated, many other and/or additional coating combinations and/or configurations can be used. The concentration of one or more biological agents, the type of polymer, the type and/or shape of surface structure and/or micro-structures in the medical device and/or the coating thickness of one or more biological agents can be used to control the release time, the release rate and/or the dosage amount of one or more biological agents; however, other or additional combinations can be used. As such, the biological agent and polymer system combination and location on the medical device can be numerous. As can also be appreciated, one or more biological agents can be deposited on the top surface of the medical device to provide an initial uncontrolled burst effect of the one or more biological agents prior to 1) the control release of the one or more biological agents through one or more layers of polymer system that include one or more non-porous polymers and/or 2) the uncontrolled release of the one or more biological agents through one or more layers of polymer system. The one or more biological agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition. The thickness of each polymer layer and/or layer of biological agent is generally at least about 0.01 μm.

In another and/or alternative non-limiting aspect of the present invention, controlled release of one or more biological agents from the medical device, when controlled release is desired, can be accomplished by using one or more non-porous polymer layers and/or by use of one or more biodegradable polymers used to at least partially form the medical device; however, other and/or additional mechanisms can be used to controllably release the one or more biological agents. The one or more biological agents can be at least partially controllably released by molecular diffusion through the one or more non-porous polymer layers and/or from the one or more biodegradable polymers used to at least partially form the medical device. When one or more non-porous polymer layers are used, the one or more polymer layers are typically biocompatible polymers; however, this is not required. One or more non-porous polymers can be applied to the medical device without the use of chemical, solvents, and/or catalysts; however, this is not required. In one non-limiting example, the non-porous polymer can be at least partially applied by, but not limited to, vapor deposition and/or plasma deposition. The non-porous polymer can be selected so as to polymerize and cure merely upon condensation from the vapor phase; however, this is not required. The application of the one or more non-porous polymer layers can be accomplished without increasing the temperature above ambient temperature (e.g., 65-90° F.); however, this is not required. The non-porous polymer system can be mixed with one or more biological agents prior to being formed into at least a portion of the medical device and/or be coated on the medical device, and/or be coated on a medical device that previously included one or more biological agents; however, this is not required. The use or one or more non-porous polymers allows for accurate controlled release of the biological agent from the medical device. The controlled release of one or more biological agents through the non-porous polymer is at least partially controlled on a molecular level utilizing the motility of diffusion of the biological agent through the non-porous polymer. In one non-limiting example, the one or more non-porous polymer layers can include, but are not limited to, polyamide, parylene (e.g., parylene C, parylene N) and/or a parylene derivative.

In still another and/or alternative non-limiting aspect of the present invention, controlled release of one or more biological agents from the medical device, when controlled release is desired, can be accomplished by using one or more polymers that form a chemical bond with one or more biological agents. In one non-limiting example, at least one biological agent includes trapidil, trapidil derivative or a salt thereof that is covalently bonded to at least one polymer such as, but not limited to, an ethylene-acrylic acid copolymer. The ethylene is the hydrophobic group and acrylic acid is the hydrophilic group. The mole ratio of the ethylene to the acrylic acid in the copolymer can be used to control the hydrophobicity of the copolymer. The degree of hydrophobicity of one or more polymers can be also be used to control the release rate of one or more biological agents from the one or more polymers. The amount of biological agent that can be loaded with one or more polymers may be a function of the concentration of anionic groups and/or cationic groups in the one or more polymer. For biological agents that are anionic, the concentration of biological agent that can be loaded on the one or more polymers is generally a function of the concentration of cationic groups (e.g. amine groups and the like) in the one or more polymer and the fraction of these cationic groups that can ionically bind to the anionic form of the one or more biological agents. For biological agents that are cationic (e.g., trapidil, etc.), the concentration of biological agent that can be loaded on the one or more polymers is generally a function of the concentration of anionic groups (i.e., carboxylate groups, phosphate groups, sulfate groups, and/or other organic anionic groups) in the one or more polymers, and the fraction of these anionic groups that can ionically bind to the cationic form of the one or more biological agents. As such, the concentration of one or more biological agent that can be bound to the one or more polymers can be varied by controlling the amount of hydrophobic and hydrophilic monomer in the one or more polymers, by controlling the efficiency of salt formation between the biological agent, and/or the anionic/cationic groups in the one or more polymers.

In still another and/or alternative non-limiting aspect of the present invention, controlled release of one or more biological agents from the medical device, when controlled release is desired, can be accomplished by using one or more polymers that include one or more induced cross-links. These one or more cross-links can be used to at least partially control the rate of release of the one or more biological agents from the one or more polymers. The cross-linking in the one or more polymers can be instituted by a number to techniques such as, but not limited to, using catalysts, using radiation, using heat, and/or the like. The one or more cross-links formed in the one or more polymers can result in the one or more biological agents to become partially or fully entrapped within the cross-linking, and/or form a bond with the cross-linking. As such, the partially or fully biological agent takes longer to release itself from the cross-linking, thereby delaying the release rate of the one or more biological agents from the one or more polymers. Consequently, the amount of biological agent, and/or the rate at which the biological agent is released from the medical device over time can be at least partially controlled by the amount or degree of cross-linking in the one or more polymers.

In still a further and/or alternative aspect of the present invention, a variety of polymers can be coated on the medical device and/or be used to form at least a portion of the medical device. The one or more polymers can be used on the medical for a variety of reasons such as, but not limited to, 1) forming a portion of the medical device, 2) improving a physical property of the medical device (e.g., improve strength, improve durability, improve biocompatibility, reduce friction, etc.), 3) forming a protective coating on one or more surface structures on the medical device, 4) at least partially forming one or more surface structures on the medical device, and/or 5) at least partially controlling a release rate of one or more biological agents from the medical device. As can be appreciated, the one or more polymers can have other or additional uses on the medical device. The one or more polymers can be porous, non-porous, biostable, biodegradable (i.e., dissolves, degrades, is absorbed, or any combination thereof in the body), and/or biocompatible. When the medical device is coated with one or more polymers, the polymer can include 1) one or more coatings of non-porous polymers; 2) one or more coatings of a combination of one or more porous polymers and one or more non-porous polymers; 3) one or more coatings of one or more porous polymers and one or more coatings of one or more non-porous polymers; 4) one or more coating of porous polymer, or 5) one or more combinations of options 1, 2, 3 and 4. The thickness of one or more of the polymer layers can be the same or different. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers that can be coated on the medical device and/or used to at least partially form the medical device can be polymers that considered to be biodegradable; polymers that are considered to be biostable; and/or polymers that can be made to be biodegradable and/or biodegradable with modification. Non-limiting examples of polymers that are considered to be biodegradable include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g. poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarate-co-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; arnylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., Nylon 66, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that considered to be biostable include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n-butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g. polystyrene); poly(vinyl ethers) (e.g. polyvinyl methyl ether); poly(vinyl ketones); poly(vinylidene halides) (e.g. polyvinylidene fluoride, polyvinylidene chloride); poly(vinylpyrolidone); poly(vinylpyrolidone)/vinyl acetate copolymer; polyvinylpridine prolastin or silk-elastin polymers (SELP); rubber; silicone; silicone rubber; polyurethanes (polycarbonate polyurethanes, silicone urethane polymer) (e.g., chronoflex varieties, bionate varieties); vinyl halide polymers and/or copolymers (e.g. polyvinyl chloride); polyacrylic acid; ethylene acrylic acid copolymer; ethylene vinyl acetate copolymer; polyvinyl alcohol; poly(hydroxyl alkylmethacrylate); polyvinyl esters (e.g. polyvinyl acetate); and/or copolymers, blends, and/or composites of above. Non-limiting examples of polymers that can be made to be biodegradable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used. The one or more polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

In another and/or alternative non-limiting aspect of the present invention, the medical device, when including and/or is coated with one or more biological agents, can include and/or can be coated with one or more biological agents that are the same or different in different regions of the medical device and/or have differing amounts and/or concentrations in differing regions of the medical device. For instance, the medical device can a) be coated with and/or include one or more biological agents on at least one portion of the medical device and at least another portion of the medical device is not coated with and/or includes biological agent; b) be coated with and/or include one or more biological agents on at least one portion of the medical device that is different from one or more biologicals on at least another portion of the medical device; c) be coated with and/or include one or more biological agents at a concentration on at least one portion of the medical device that is different from the concentration of one or more biological agents on at least another portion of the medical device; etc.

In still another and/or alternative non-limiting aspect of the present invention, one or more portions of the medical device can 1) be formed of the same or different materials, 2) include the same or different biological agents, 3) include the same or different amounts of one or more biological agents, 4) include the same or different polymer coatings, 5) include the same or different coating thicknesses of one or more polymer coatings, 6) have one or more of both sections controllably release and/or uncontrollably release one or more biological agents, and/or 7) have one or more portions of one section controllably release one or more biological agents and one or more portions of the other section uncontrollably release one or more biological agents.

In another and/or alternative embodiment of the invention, the medical device can be used in a procedure to retrieve a treatment device. During the course of a medical treatment, problems with a particular medical treatment can arise such as, but not limited to, 1) a guide wire become damaged, thus interfering with the proper placement of a medical device in a body passageway and/or inability to properly position the guide wire in a body passageway, 2) a treatment device becomes improperly positioned on another treatment device (e.g., stent dislodges from angioplasty balloon, etc.), 3) the treatment device is damaged (e.g., stent is bent, angioplasty balloon torn, etc.), 4) a treatment device does not disengage from another treatment device (e.g., angioplasty balloon gets stuck to stent, etc.) and/or 5) the size of the treatment device needs to be changed (e.g., stent too small, stent too large, angioplasty balloon too small, etc.). In many of these situations, the guide wire, treatment device and/or guide catheter has to be partially for fully removed from the patient before the problem can be solved. The removal of these devices not only loses the treatment site that was obtained by the previously inserted devices, but the removal and subsequent reinsertion of the guide catheter, guide wire and/or treatment device to a treatment site results in significantly increased time and cost for the medical procedure, can increase the risk of damage to a body passageway of a patient and/or can increase the health risk of the patient during and/or after the medical procedure. The use of the medical device of the present invention can be used to overcome such past problems. The medical device can be used to retain the location of the treatment site that was obtained by the guide catheter, thus reducing the time and cost of the medical procedure, reducing the risk of damage to a body passageway of a patient and/or reducing the health risk of the patient during and/or after the medical procedure. The medical device can also or alternative be used to reduce damage to one or more treatment devices and/or the body passageway during the correction of one or more of these medical situations. For instance, during a particular medical procedure, a guide catheter is manipulated close to a diseased area of a body passageway. The diseased area is commonly located in a body passageway that is narrower than the diameter or cross-sectional area of the guide catheter. As such, the guide catheter is positioned as close to the diseased area as possible. It is not uncommon for the end of the guide catheter to be positioned several inches from the diseased area. It is also not uncommon for the guide catheter to be inserted into the body passageway until the end of the guide catheter mostly or fully impairs fluid flow through the body passageway. One the guide catheter is positioned in the body passageway, a guide wire is commonly fed through the guide catheter and then fed through or across a diseased area of a body passageway. A treatment device (e.g., angioplasty balloon with or without a stent) is then tracked over or fed along the guide wire and into position for treatment of the diseased area. If the guide wire becomes damaged, and/or the stent and/or angioplasty balloon needs to be replaced, the medical device of the present invention is can be inserted into and through the guide catheter and to a point closely adjacent to the diseased or treatment area of a body passageway. When the medical device is used prior to the removal of one or more treatment devices from the body passageway, the front end of the medical device can be positioned at or near the treatment site or diseased area, thus preserving the treatment site for later procedures.

In still another and/or alternative embodiment of the invention, the medical device of the present invention can be used in a procedure to retrieve a treatment device that has disassociated from another treatment device (e.g., stent becoming disconnected from an angioplasty balloon, etc.). In this situation, the treatment device must be removed from the body passageway. In the past, a snare was used to grab the treatment device and withdraw the treatment device into the guide catheter for final removal from the body. One problem associated with this complicated medical procedure is that the withdrawal of the stent through the body passageway can cause the medical device to scrape up against or otherwise damage a body passageway during the withdrawal process. This past problem can be overcome by the use of the medical device of the present invention. Prior to, during or after the treatment device has been grasped by the snare, the medical device can be inserted through the guide catheter and adjacent to the treatment device. Thereafter, the treatment device can be withdrawn through the medical device and into the guide catheter. As can be appreciated, the guide catheter can be removed prior to drawing the treatment device through the medical device. In either situation, the sides of the medical device protect the body passageway from damage from the treatment drive as the treatment device is removed from the body passageway. The front end of the medical device also is maintained at or near the diseased area, thus a new treatment device can be simply fed through the medical device and to the diseased area of the body passageway.

In yet another and/or alternative embodiment of the invention, the medical device of the present invention can be used in a procedure to insert a treatment device to a diseased area of a body passageway and minimize the period of time fluid flow through the body passageway is disrupted. In certain medical procedures, blood flow through a vein or artery needs to be disrupted for a period of time to fix the impaired flow or blockage. The termination of blood flow can be dangerous to the patient. In the past when a delay or problem occurred, the guide catheter had to be partially or fully retracted from a treatment site to allow for blood flow through the vein or artery. The withdrawal of the guide catheter resulted in increased time and cost associated with the medical procedure due to loss of positioning of the treatment site, and also could increase te health risk a patient due to increased treatment times and/or having to reposition the guide catheter at the treatment area. This past problem is overcome by the use of the medical device of the present invention. The medical device can be inserted through the guide catheter. Thereafter, the guide catheter can be partially retracted into a larger artery, thereby allowing blood to flow through the artery. Since the medical device has a small diameter or cross-sectional area than the guide catheter, blood is able to also flow around the medical device. When the guide catheter needs to be repositioned at the treatment site, the guide catheter can be simply guided to the treatment side by feeding the guide catheter along the medical device. As such, significant time is saved for the repositioning of the guide catheter. The treatment device can then be fed to the diseased area through the medical device, or the medical device can be removed from the guide catheter and then the treatment device can then be fed to the diseased area through the guide catheter. As can be appreciated, the one or more slots in the medical device can be used to facilitate in the flow of blood or other fluids through the medical device, thereby reducing the occurrence of the blood or other fluids from being occluded. As can also or alternatively be appreciated, the medical device can include a plurality of openings or perforations to facilitate in the flow of blood or other fluids through the medical device.

In still yet another and/or alternative embodiment of the invention, the medical device of the present invention can be used in a procedure to protect a fragile treatment device from being damaged while delivering the treatment device to a treatment site and/or to prevent a treatment device from damaging healthy tissue while delivering the treatment device to a diseased area of a body passageway. This medical procedure is accomplished by simply at least partially covering the treatment device with the medical device of the present invention. The treatment device can be at least partially covered by the medical device of the present invention prior to inserting the treatment device in the guide catheter, after the treatment device has been inserted into the guide catheter but prior to the treatment device being fully ejected from the front end of the guide catheter, or after the treatment device has been ejected from the front end of the guide catheter.

One object of the present invention is the provision of a medical device that improves procedural success rates of inserting a treatment device into a body passageway.

Another and/or alternative object of the present invention is the provision of a medical device that can be used to replace of treatment device without losing the treatment site that was obtained by the guide catheter.

Still another and/or alternative object of the present invention is the provision of a medical device that at least partially protect a body passageway during the insertion and/or removal of a treatment device.

Still yet another and/or alternative object of the present invention is the provision of a medical device that can at least partially protect a treatment device during the insertion of the treatment device to a diseased area of a body passageway.

A further and/or alternative object of the present invention is the provision of a medical device that can allow fluid flow through a body passageway during a medical procedure used to insert a treatment device in a diseased area of a body passageway.

These and other advantages will become apparent to those skilled in the art upon the reading and following of this description taken together with the accompanying drawings.

### BRIEF DESCRIPTION OF THE DRAWINGS

Reference may now be made to the drawings, which illustrate various embodiments that the invention may take in physical form and in certain parts and arrangements of parts wherein:

FIG. 1 is an elevation view of a prior art guide catheter and insertion tool;

FIG. 2 is a diagram illustrating the heart and main arteries of a human and a guide catheter being inserted into one of the arteries;

FIG. 3 is an elevation view of a heart having several patches of atheroma in the coronary artery and also illustrating the portioning of a guide catheter near one patch of atheroma in the coronary artery;

FIG. 4 is a cross-section view of a section of the coronary artery illustrated in FIG. 3 that includes a patch of atheroma and the insertion of an angioplasty balloon that is covered by a stent into the diseased area of the coronary artery;

FIG. 5 illustrates the expansion of the angioplasty balloon and expansion of the stent in of the coronary artery of FIG. 4;

FIG. 6 illustrates the deflation and removal of the angioplasty balloon from the expanded stent that is left in the coronary artery;

FIG. 7 is a cross-section view of the medical device in accordance with the present invention positioned within a guide catheter;

FIG. 8 is an elevation view of a section of the medical device of the present invention;

FIG. 9 is an elevation view of a guiding hub that can be connected to an end of the medical device of FIG. 8;

FIG. 10A illustrates the snagging of an angioplasty balloon on a stent that has been expanded in a blood vessel;

FIG. 10B illustrates damage to an expanded stent while a snagged angioplasty balloon is pulled from the stent;

FIGS. 11A and 11B illustrated the use of the medical device of FIG. 8 to remove a snagged angioplasty balloon from a stent without damaging the stent;

FIGS. 12A and 12B illustrated the inadvertent detachment of a stent from an angioplasty balloon and the prior art procedure for recovering the detached stent;

FIGS. 13A-13C illustrate the use of the medical device of FIG. 8 to recover an inadvertently detached stent;

FIGS. 14A and 14B illustrate a prior art angioplasty procedure wherein the end of the guide wire is bent after passing through a first diseased area in a blood vessel;

FIGS. 15A-15C illustrates the use of the medical device of FIG. 8 to retrieve a damaged guide wire and to guide a stent to a second diseased area in a blood vessel;

FIG. 16 illustrates the obstruction of a stent by small deposits in a blood vessel thereby preventing the stent being placed in a diseased area in the blood vessel; and,

FIGS. 17A and 17B illustrate the use of the medical device of FIG. 8 to guide a stent about deposits in a blood vessel so as to enable the stent to be place in a diseased area in the blood vessel.

### DETAILED DESCRIPTION OF THE INVENTION

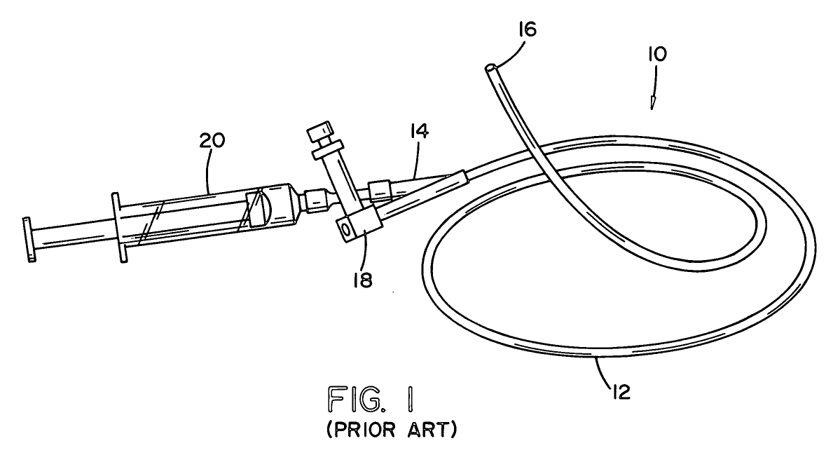

Referring now to the drawings wherein the showing is for the purpose of illustrating preferred embodiments of the invention only and not for the purpose of limiting the same, FIGS. 1-6 illustrate prior art mechanical devices that are used in an angioplasty procedure to repair a diseased artery. A prior art angioplasty procedure can include the used of these prior art mechanical devices to physically open a clogged vascular structure. These mechanical devices include stents that are either balloon expandable or self expanding. Although these procedures are technically common, various adverse events can occur during this medical procedure such as, but not limited to, a) the stent sticking or snagging on the angioplasty balloon, b) the stent becoming dislodged from angioplasty balloon prior to final deployment of the stent, c) the guide wire being damaged during the insertion of the guide wire through a blood vessel, d) the inability of the angioplasty balloon and/or stent to be maneuvered about deposits in a blood vessel, etc. Furthermore, there may arise the need to exchange or replace an angioplasty balloon, stent and/or guide wire during a medical procedure. When any of these events occur, the guide catheter typically must be partially or fully removed from a treatment site to allow for fluid flow through the body passageway. This is typically required when the treatment device is used in the cardiovascular system. Blood flow cannot be interrupted for extended periods of time without risk of injury to the patient. As such, when a complication occurs and/or treatment modification occurs thereby increasing the treatment time, the guide catheter typically is retracted from a treatment site to allow for blood to temporarily flow through the artery during the extended procedure. As such the guide catheter position is lost and requires additional time to reinsert the guide catheter in position so that the corrective and/or modified treatment can begin again. As will be described in more detail below, the medical device of the present invention can be used to reduce such treatment times during a corrective or modified treatment. The medical device of the present invention can also improved the success rate of a particular treatment and/or reduce risk or damage to a patient during the medical treatment.

FIG. 1 illustrates a prior art guide catheter device 10 that includes a tubular guide catheter 12. The tubular catheter is typically formed of a flexibly rubber or polymer material; however, other or additional materials can be used. The back end of the guide catheter is connected to the front end of a Y-shaped device 14. One back end of the Y-shaped device includes a one-way valve or opening arrangement 16 that enables treatment devices such as guide wires, angioplasty balloons, stents, etc. to be inserted therethrough and into the guide catheter. The other back end of the Y-shaped device can be connected to a fluid insertion device 20. The fluid insertion device allows medicine, saline fluid, etc. to be inserted into the guide catheter. These mechanical devices are well known in the art, thus will not be described in detail. The length and diameter of the guide catheter is selected to enable a physician to insert the guide catheter in close proximity to a diseased area of an artery. Typically the tubular guide catheter is about 50-142 cm (19.5-56 inches); however, other lengths can be used.

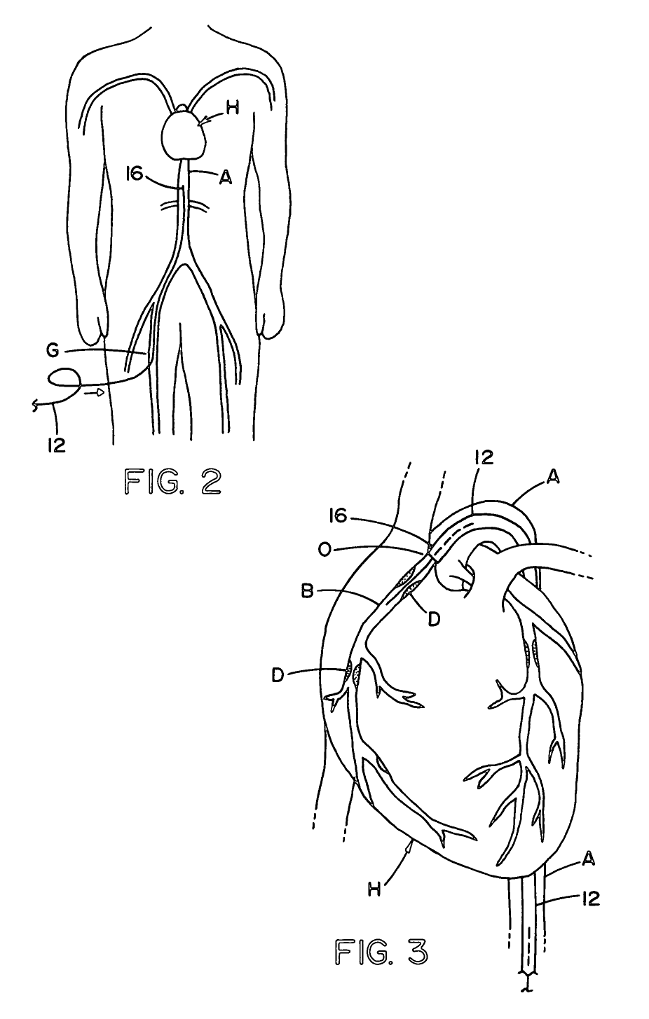

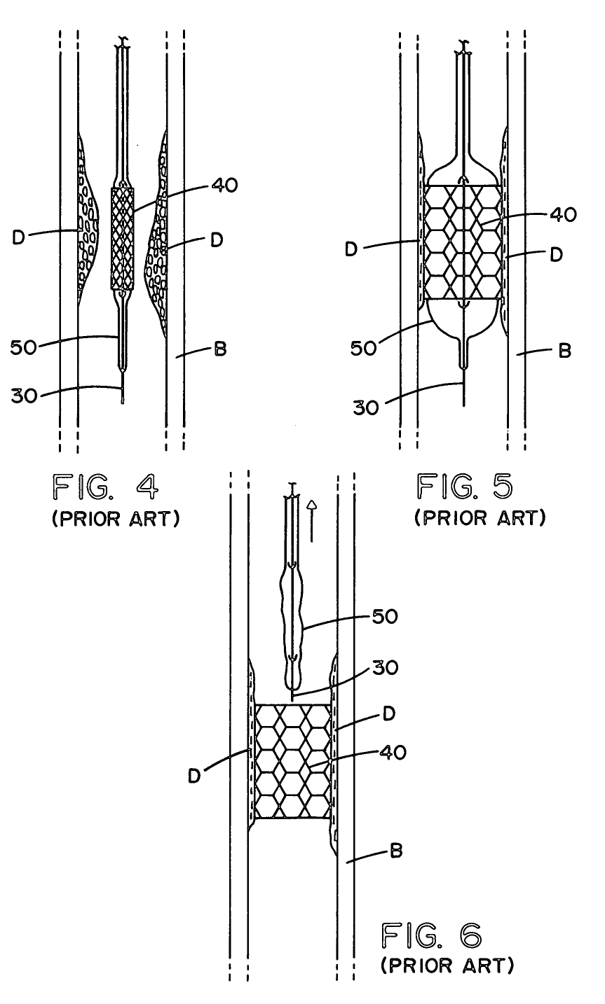

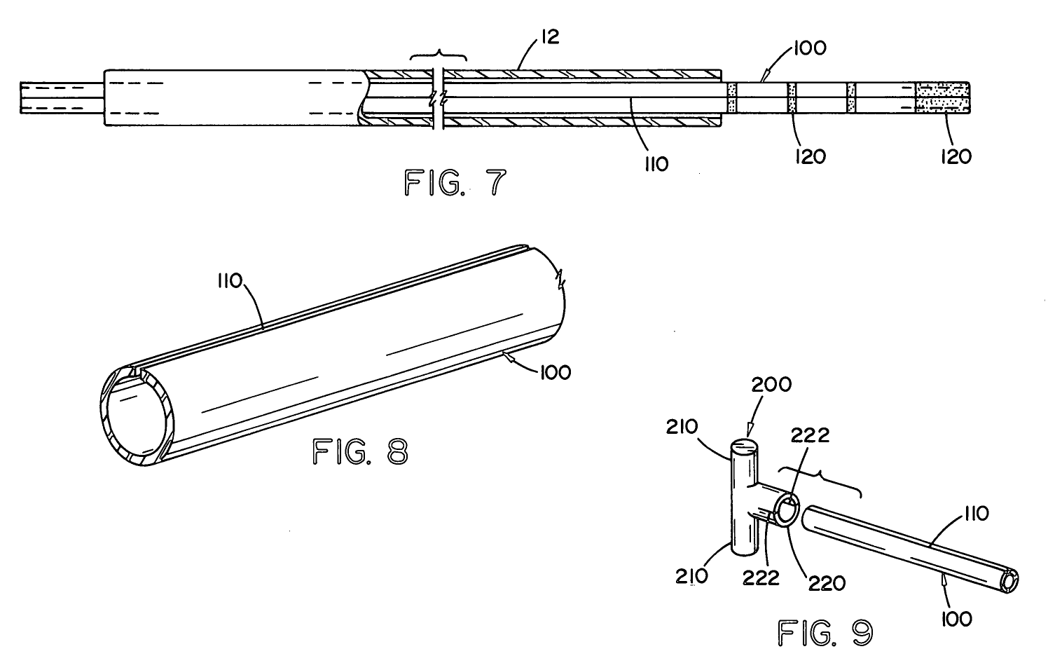

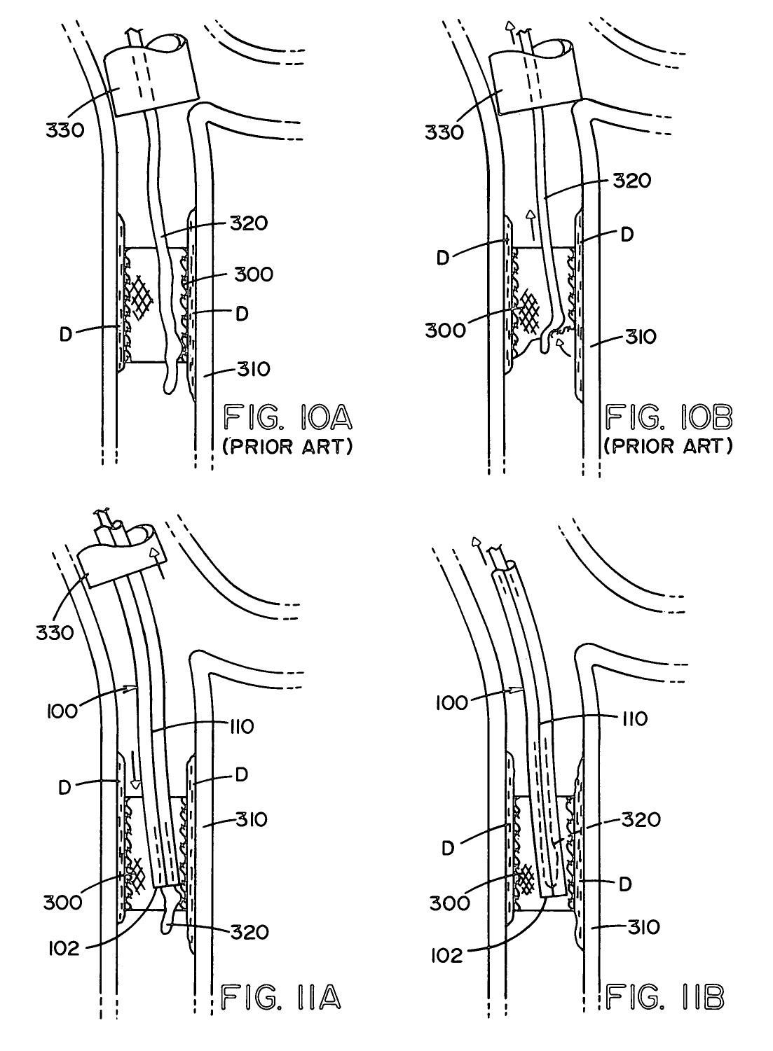

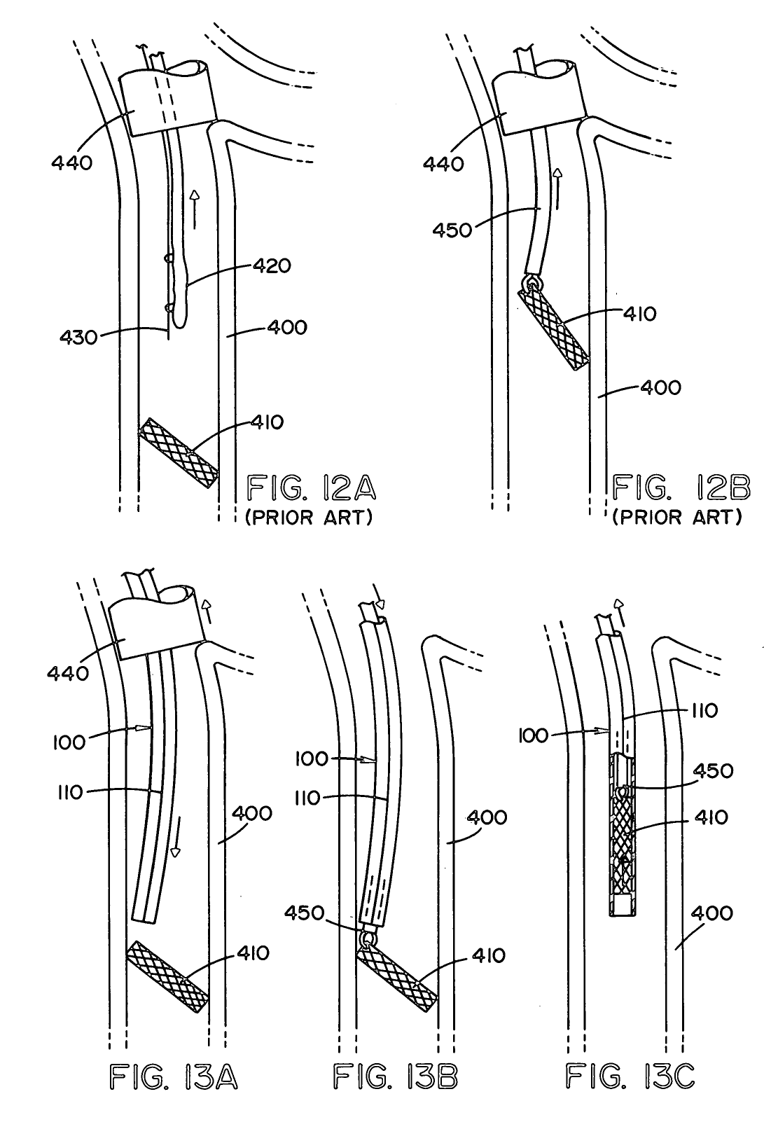

As illustrated in FIG. 2, the guide catheter 10, when used in a vascular system, is commonly inserted into a blood vessel in the groin G. The front end 16 of the guide catheter is inserted into the large blood vessels (e.g., aorta) of the heart H. As illustrated in FIG. 3, the front end of the guide catheter is fed through aorta A and to the ostium O of the right coronary artery B of heart H that includes patches of atheroma. As can be appreciated, the diseased area can be located in other or additional blood vessels in the heart and/or other regions of the body. As illustrated in FIG. 3, the diameter of the tubular guide catheter 12 is larger than the diameter of the blood vessel B. As such, the front end 16 of the tubular guide catheter can only be advanced to the ostium of blood vessel B. The ostium of the blood vessel B typically is referred to as the treatment area or treatment region. When front end 16 of the tubular guide catheter is positioned against the ostium, the blood flow through blood vessel B is reduced or terminated. Fluid flow through blood vessel B can be regulated by inserted fluids through the tubular guide catheter via fluid insertion device 20. Typically the front end 16 of the guide catheter is positioned within about 0.1-10 inches of a diseased area, and typically within about 6 inches of a diseased area of the artery; however, other distances can be used. Once the guide catheter is positioned in a treatment area, a guide wire 30 is fed through one end of the Y-shaped device and into and through the guide catheter and then in or through the diseased area of the blood vessel. As can be appreciated, the guide wire can be partially or fully threaded in the guide catheter prior to the guide catheter being positioned in the treatment area.

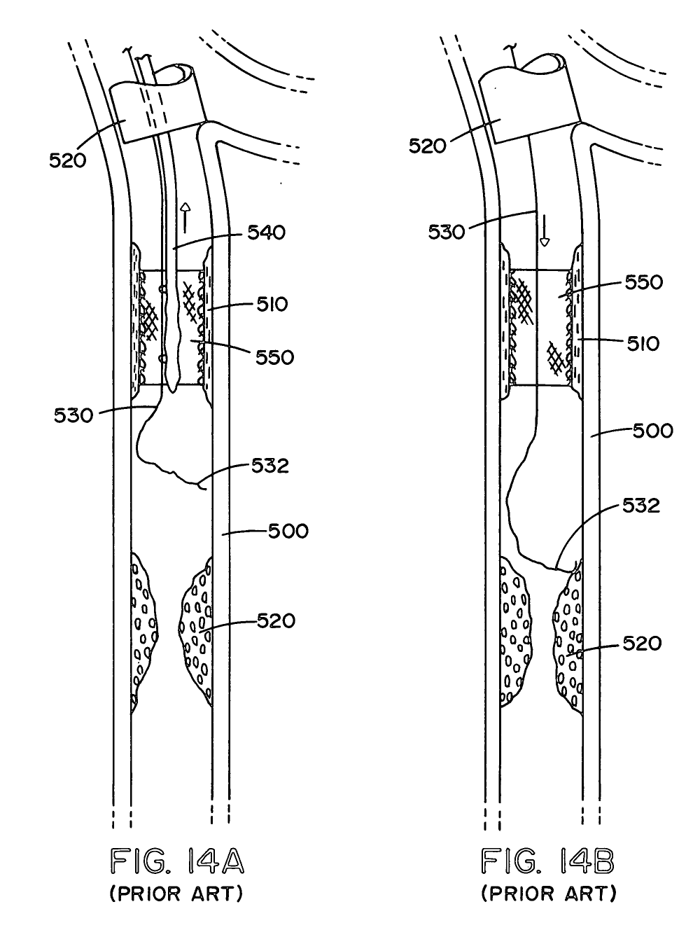

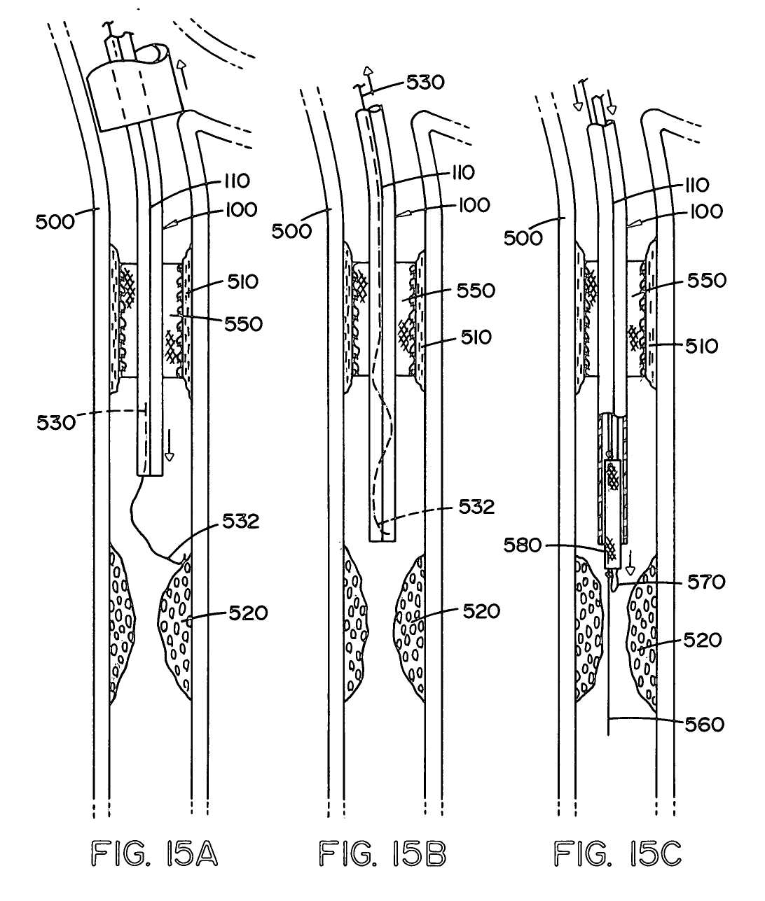

Once the front end of the guide wire has be positioned to or at least partially through the diseased area in the blood vessel, an angioplasty balloon 50 or a stent 40 crimped on an angioplasty balloon 50 is guided along the guide wire to the diseased area D of blood vessel B as illustrated in FIG. 4. Once the stent and angioplasty balloon are positioned in the diseased area D, the angioplasty balloon is expanded thereby causing the stent to deform and expand as illustrated in FIG. 5. The deformation of the stent in the blood vessel compresses the atheroma in the blood vessel thereby unblocking or widening the opening through the blood vessel to enable better blood flow through the blood vessel. After stent 40 has been expanded, angioplasty balloon 50 is deflated and guide wire 30 and angioplasty balloon 50 are retracted from blood vessel B as illustrated in FIG. 6. The stent remains in the blood vessel to facilitate in the blood flow through the blood vessel.

Referring now to FIGS. 7 and 8, a medical device 100 in accordance with the present invention is illustrated. The medical device 100 has a generally tubular shape with a generally constant diameter along a longitudinal length of the medical device; however, the medical device can have other shapes and/or non-constant cross-sectional shapes along the longitudinal length of the medical device. The outside diameter of the medical device is designed to be less than the inner diameter of the guide catheter as illustrated in FIG. 7. The smaller outer diameter of the medical device enables the medical device to be fed through a guide catheter. Typically the cross-sectional area of the medical device is at least about 2% less than the cross-sectional area of the inner passageway of the tubular guide catheter, and more typically about 5-50% less than the cross-sectional area of the inner passageway of the tubular guide catheter. As can be appreciated, other cross-sectional area sizes of the medical device can be used.

As illustrated in FIG. 8, the medical device includes a slit 110 that extends along the longitudinal length of the medical device. Typically, the slit extends along the complete longitudinal length of the medical device; however, this is not required. Typically, the slit extends over a majority of the longitudinal length of the medical device. The slit is designed to a) enable a treatment device to be moved through an internal passageway of the medical device, b) enable the medical device to at least partially fit about a treatment device such as, but not limited to stent 40 and/or angioplasty balloon 50, and/or c) enable the medical device to be fit about a guide wire and/or other treatment device that is at least partially in the guide catheter. The medical device can be used in a variety of ways such as, but not limited to, function in part as a sleeve that can be insertable about one or more treatment devices, used to retain a treatment site when a guide catheter has be removed or repositioned in a body passageway, etc.

Referring again to FIG. 7, one or more regions of the medical device 100 can include a marker 120 that can be used to facilitate is viewing and/or recording the movement and/or position of the medical device during a medical procedure. The markers can be coated on the medical device (e.g., polymer coating that includes a marker, etc.), bonded to the medical device (e.g., bonded metal bands, etc.) and/or forms part of the medical device (e.g., metal plastic that is formed into part of the medical device, etc.). The one or more markers on the medical device can also be used to facilitate in measuring the length of the diseased area. For instance, the medical device can include one or more radiopaque marker positioned at least at the distal tip of the medical device to facilitate in guiding the medical device in a body passageway. The medical device could also or alternatively include a plurality of markers (e.g., radiopaque markers, etc.) positioned along select regions on the longitudinal length of the medical device so that the medical device can be used as a ruler to measure the length of a diseased area. As can be appreciated, the markers can be used for other or additional reasons.

The design of the medical device of the present invention can enable a physician to 1) improve the success of angioplasty procedures; 2) reduce procedure times and/or patient risk when a) a treatment device had to be replaced, b) a treatment device had to be retrieved, c) a fragile treatment device is used, d) a diseased region is difficult to access, and/or e) one or more treatment devices does not properly engage and/or disengage fro one another; and/or 3) reduce trauma and/or damage to body passageways during a medical procedure. As can be appreciated, the medical device can be used for additional or alternative purposes. As can also be appreciated, the medical device can be used in body passageways other that the vascular system.

The medical device 100 is typically formed of a flexible material so that the medical device can be fed through a guide catheter. As can be appreciated, different regions of the medical device can have differing flexibility; however, this is not required. The medical device can be at least partially formed from one or more materials such as, but not limited to, plastic or other polymers, metals, combined polymers and plastics, fiber reinforced polymers (e.g, fiberglass fibers embedded in polymers, Kevlar fibers embedded in polymers, etc.) and combinations thereof. One or more regions of the medical device can be strengthened, reinforced, and/or stiffened by the use of one or more materials. The materials used to strengthen, reinforce, and/or stiffen one or more portions of the medical device can be incorporated in the material or materials forming the medical device (e.g.; mixed with, etc.) and/or connected to the medical device (e.g., a reinforcement sleeve inserted about the exterior and/or interior of the medical device, etc.). The medical device can be coated and/or impregnated on one or more regions of the exterior and/or interior of the medical device to a) facilitate in the insertion of the medical device in a guide catheter, b) to facilitate in the insertion of the medical device about a treatment device, c) to reduce the friction of one or more surfaces of the medical device, and/or d) to reduce or eliminate rough and/or sharp surfaces on the medical device. The coating and/or impregnated portions on the medical device can also or alternatively include one or more biological agents. The coating thickness on the one or more regions of the medical device can be uniform or vary in one or more regions of the medical device. The same or different coatings can also be used on one or more regions of the medical device. The coating thickness on the medical device is controlled so that the outer diameter of the medical device can be inserted through the interior passageway of a guide catheter. The coating thickness can be uniform or vary in different regions of the medical device.

As illustrated in FIGS. 7-8, the medical device includes at least one slit 110. This slit is at least about 1% of the longitudinal length of the medical device, and typically at least about 25% of the longitudinal length of the medical device, and more typically at least about 50% of the longitudinal length of the medical device, and still more typically at least about 75% of the longitudinal length of the medical device. As shown in FIG. 7, slit 110 extends about 100% of the longitudinal length of the medical device. The width of the slit can vary depending on a particular application of the medical device. As can be appreciated, the edges of the slit can overlap, be spaced apart, or contact one another. The one or more slits 110 in the medical device are typically generally straight as illustrated in FIGS. 7 and 8; however, the slit can have other shapes (e.g., zig zag shapes, wave shapes, etc.). The medical device can also include one or more secondary slits. These secondary slits can be less than 1% or greater than or equal to 1% of the longitudinal length of the medical device. These secondary slits can be used for various purposes, such as, but not limited to, enabling fluid flow through the medical device, enabling leaching, etc.; however, other or additional uses of the secondary slits can be appreciated.

The medical device typically has a longitudinal length that is at least about 10% of the longitudinal length of a guide catheter. The medical device can be designed to be longer than the guide catheter so that the medical device can be fed completely through the guide catheter and extend out from the guide catheter to a diseased site. It is not uncommon for a diseased sited to be located about 0.1-10 inches or more from the front end of the guide catheter. The longitudinal length of the medical device can be designed to be several inches longer than the guide catheter (i.e., over 100% the length of the guide catheter tube) to enable the medical device to be fed through the guide catheter and to a disease site that is several inches from the front end of the guide catheter and/or to enable a physician to manipulate the back end of the medical device during a medical procedure. As such, the medical device could be as much as 200% or more the length of the tubular guide catheter. The material used to form the medical device is generally designed to maintain the natural shape of the medical device. As such, when the shape of the medical device is altered, such as when a treatment device is pulled through the front end and/or through the interior passageway of the medical device, the shape of the medical device substantially reforms to its natural shape once the treatment device has pass through a particular region of the medical device; however, this is not required.

Referring now to FIG. 9, a gripping device or guide hub 200 can be used with the medical device. The guide hub can include one or more gripping members 210 to facilitate in the movement or manipulation of the medical device during a medical procedure. As can be appreciated, many other designs and/or configurations of the gripping member can be used. One end of the guide hub includes a connector 220. As illustrated in FIG. 9, the connector includes a slit 222 that facilitates in the connecting of one end of the medical device to the guide hub. As can be appreciated, the slit can also designed to enable the guide hub to fit about a treatment device (e.g., guide wire, etc.) that is positioned at least partially in the guide catheter. As can be appreciated, many other or additional connection arrangements can be used. The connection arrangement can be also designed to enable the medical device to be releaseably connected to the guide hub. The guide hub can be made of a variety of materials such as, but not limited to, rubber, plastic, metal, polymeric materials or any combination thereof. One or more regions of the hub can be stiff, flexible, etc. The guide hub can be utilized to withdraw and/or advance the medical device in a guide catheter and/or body passageway. The guide hub can be attached to a device and/or include an opening that allows for 1) the injection of fluids in the interior passageway of the medical device, and/or 2) the insertion and/or removal of one or more treatment devices from the medical device. As such, the medical device of the present invention can be designed to enable the medical device to be partially or fully inserted about one or more treatment devices (e.g., stent, angioplasty balloon, guide wire, etc.) and/or to enable one or more treatment devices to be moved in the internal passageway of the medical device. The one or more slits on the medical device can be used to facilitate in enabling one or more treatment devices to be moved within the internal passageway of the medical device and/or facilitate in enabling the medical device to be at least partially inserted about a treatment device. The insertion of the medical device about one or more treatment devices can be accomplished by physical manipulation of the medical device and/or by use of an insertion tool (e.g., guide hub, etc.). The insertion tool, when used, can be used to facilitate in the manipulation of the medical device (e.g., cause the front end of the medical device to open so as to at least partially capture and/or release a treatment device, cause the medical device to move into a particular blood vessel, cause the medical device to move about one or more obstructions in a blood vessel, cause the front end of a medical device to be positioned near or at least partially about a treatment device, etc.).

As set forth above, the medical device can be used for a variety of purposes such as, but not limited to, a) maintaining a treatment site when the guide catheter needs to be partially or fully retracted from a body passageway, b) protecting a treatment device from damage, c) inhibiting or preventing a treatment device from damaging a body passageway, d) facilitating in the insertion of a treatment device to a diseased location, e) facilitating in the retrieval of a treatment device, and/or f) facilitating in fluid flow through narrow body passageways. Several of these uses of the medical device are illustrated in FIGS. 11A-11B, 13A-13C, 15A-15C and 17A-17B. A brief discussion of a few of the potential uses of the medical device are set forth below.

Referring now to FIGS. 10A and 10B, there is illustrated an expanded stent 300 in blood vessel 310. The expanded stent has opened a blockage in a diseased area D of the blood vessel. A deflated angioplasty balloon 320 is shown extending out from the end of guide catheter 330. The front end of the guide catheter is positioned at the ostium of the blood vessel. An end portion of the angioplasty balloon 320 is hooked on or snagged to the stent. In the past, the common procedure to remove the angioplasty balloon from the stent was to pull the angioplasty balloon as illustrated by the arrow until the angioplasty balloon released from the stent. The pulling of the angioplasty balloon increased the risk that the balloon would tear and potentially allow air bubbles into the blood vessel. The pulling of the angioplasty balloon from the stent can also result in the balloon damaging and/or deforming the stent so that it does not properly function. This damaging of the stent is illustrated in FIG. 10B. The damaged stent can result in structural damage to the stent which could allow the blood vessel to compress the stent and reform the blockage in the blood vessel. The damaged stent may also ro alternatively damage the blood vessel which can result in clotting in the blood vessel, piercing or weaken of the blood vessel, etc. The damaged stent may also block other angioplasty balloons and/or stents from passing the damaged stent, thus impairing medical treatment to diseased regions in the blood vessel that are upstream from the damaged stent. The pulling of the angioplasty balloon from the stent can also cause the stent to be come dislodged in the blood vessel.

As shown in FIGS. 11A and 11B, the medical device 100 of the present invention can be used to facilitate in releasing or disengaging the angioplasty balloon from the stent. Medical device 100 is inserted about the balloon at the back end of the guide catheter and then fed through the guide catheter and to the interior surface of the stent where the angioplasty balloon is snagged. At this point, the guide catheter can be retracted if blood flow through the blood vessel is required as illustrated by the arrow. By positioning the end of the medical device near the snagged location, the medical device can be used to release the snagged balloon. For instance, the front end 102 of the medical device can be slightly depressed against the side of the stent to cause the release of the angioplasty balloon and the angioplasty balloon can then be pulled through the medical device as illustrated in FIG. 11B. If the angioplasty balloon does not release, the end of the medical device can be placed against the inner surface of the stent and the angioplasty balloon can then be tugged until the angioplasty balloon releases from the stent. In this particular procedure, the front end of the medical device facilitates in supporting the stent so that while the angioplasty balloon is being pulled, the stent is not dragged along the blood vessel and/or is damaged or deformed. After the angioplasty balloon is released from the stent, the angioplasty balloon can be withdrawn through the medical device as illustrated by the arrow in FIG. 11B and the medical device can be withdrawn through the guide catheter. Prior to withdrawing the medical device through the guide catheter, another angioplasty balloon could be, but is not required to be, inserted through the medical device and to the stent. Alternatively or additionally, the guide catheter can be repositioned to the treatment area by sliding the guide catheter on the medical device. Thereafter, the medical device could be withdrawn prior to inserting the angioplasty balloon or the medical device could be left in place and the angioplasty balloon can then be inserted through the medical device. The angioplasty balloon could then be inflated to ensure that the stent is properly expanded in the blood vessel. The balloon can then be deflated and removed. Thereafter, the medical device and/or guide catheter can be removed. As can be appreciated, modifications to this medical procedure can be used and/or other medical procedures can be used.

FIGS. 12A and 12B illustrate an unexpanded stent 410 in a blood vessel 400 that has separated from the angioplasty balloon 420. The angioplasty balloon is still located near the end of guide wire 430 and is shown to be extending from the end of guide catheter 440. The front end of the guide catheter is positioned at the ostium of the blood vessel. In prior art medical procedures used to retrieve the stent, the guide wire and angioplasty balloon were first removed from the guide catheter as indicated by the arrow in FIG. 12A. The guide catheter may also have to be retracted in the blood vessel to allow for blood flow through the blood vessel prior to again attempting to retrieve stent 410. A grasping device 450 illustrated in FIG. 12B was then fed through the guide catheter and was used to grasp the stent and pull the stent through the guide catheter as indicated by the arrow. Thereafter, the guide catheter was repositioned at the treatment site in the blood vessel and the angioplasty procedure was repeated.

During the recovery procedure of the stent, the dragging of the stent back to the guide catheter could cause damage to the blood vessel. The need to keep the guide catheter at or near the treatment site during the recovery procedure also could resulted in extended periods of time of interrupted blood flow through the blood vessel which could cause problems for the patient. When the guide catheter was retracted from the treatment site during the recovery procedure, a significant mount of time was typically required to reposition the guide catheter at the treatment site so that the angioplasty procedure can be completed.

Referring now to FIGS. 13A-13C, medical device 100 can be used to address the problems of retrieving stent 410 as set forth above. When the stent 410 becomes detached from the angioplasty balloon, the angioplasty balloon is retracted through the guide catheter. The guide wire can also be retracted through the guide catheter; however, this is not required. The medical device can then fed through the guide catheter and close to dislodged stent 410 as illustrated in FIG. 13A. Thereafter, the guide catheter can be retracted to allow blood flow through the blood vessel as indicated by the arrow in FIG. 13A; however, this is not required. After the medical device is positioned near stent 400, a grasping device 450 is fed through the medical device and to the stent as illustrated by the arrow in FIG. 13B. The grasping device can then grasp the stent and then withdraw the stent through the medical device as indicated by te arrow illustrated in FIG. 13C. Since the medical device is positioned closed to the dislodged stent, the chance or amount of damage to the blood vessel during the drawing of the stent into medical device is significantly reduced. Once the stent is in the inner passageway of the medical device, the walls of the medical device protect the blood vessel from damage that could be caused by the stent. The one or more slits in the medical device can be used to facilitate in the stent entering the front end of the medical device and/or facilitate in the stent moving within the interior passageway of the medical device. After the dislodged stent is removed, a guide wire can be fed through the medical device and to the diseased area. Thereafter a new angioplasty balloon and stent can be fed on the guide wire to the diseased area of the blood vessel. If the guide catheter has to be repositioned to the treatment area, the guide catheter can simply and quickly be fed along the medical device to the treatment area. As a result, the use of the medical device can save significant time in the recovery of the dislodged stent and the reinsertion and placement of a new stent. As can be appreciated, once the guide catheter is repositioned back to the treatment area, the medical device can be removed from the guide catheter prior to reinserting the guide wire, new angioplasty balloon and/or new stent. As can be appreciated, modifications to this medical procedure can be used and/or other medical procedures can be used.

Referring now to FIGS. 14A and 14B, a blood vessel 500 includes two diseased areas 510 and 520. The guide catheter 520 has been advanced to the treatment area at the ostium of the blood vessel. A guide wire 530 has been fed through the guide catheter and through diseased area 510. As illustrated in FIG. 14A, the front region 532 of the guide wire was bent and/or damaged when it was passed through diseased area 510. Since the end of the guide wire was successfully pass through the first diseased area 510, an angioplasty balloon 540 and stent 550 are fed along the guide wire to the diseased area, and then the angioplasty balloon and stent are expanded to open the first diseased area. Thereafter, the angioplasty balloon was typically retracted through the guide catheter so that a new stent crimped on a new angioplasty balloon could be fed through the guide catheter and to the second diseased area. However, since the front end of the guide wire is bent and/or damaged, the guide wire cannot be passed through the second diseased area 520 as illustrated in FIG. 14B. As such, in past medical procedures, the guide wire was fully retracted through the guide catheter as shown by the arrow in FIG. 14A and a new guide wire was inserted through the guide catheter and then through the second diseased area. The retraction of the guide wire could result in damage to the blood vessel from the end of the guide wire scratching the inner surface of the blood vessel. In addition, due to the delay in having to change out the guide wire, the guide catheter typically had to be retracted from the treatment area so that blood flow could resume flowing through the blood vessel. As such, the treatment area of the guide catheter was commonly lost. At a later time, the guide catheter was moved back to the treatment area, a procedure that took time. Thereafter, a new guide wire was inserted through the guide catheter and through the second diseased area so that a angioplasty balloon and stent could be expanded to repair the second diseased area.

Referring now to FIGS. 15A-15C, medical device 100 is used to reduce the time of the medical procedure and reduce the occurrence of damage to the blood vessel. After the stent is expanded in the first diseased area, the angioplasty balloon is deflated and retracted through the guide catheter as illustrated in FIG. 14A. Thereafter, the medical device is fed through the guide catheter and to the end of the damaged end of the guide wire as illustrated by the arrow in FIG. 15A. The damaged guide wire can then be then retracted through the medical sheath as illustrated by the arrow in FIG. 15B. During the retraction of the guide wire, the medical sheath protects the walls of the blood vessel from damage from the guide wire as it is removed from the blood vessel. The guide catheter can then be retracted to allow blood to again flow through the blood vessel as illustrated by the arrow in FIG. 15A; however, this is not required. When the medical procedure is to continue to repair the second diseased area, the guide catheter can be easily and quickly moved back to the treatment area by feeding the guide catheter along the medical device. Thereafter, the medical device can be removed from the guide catheter, or the medical device can be left in the guide catheter and a new guide wire 560 can then be fed through the second diseased area as illustrated in FIG. 15C. The new angioplasty balloon 570 and new stent 580 are fed along the guide wire to the second diseased area to repair the diseased area as illustrated by the arrow in FIG. 15C. As such, the medical device can reduce the treatment time and/or reduce damage to the blood vessel. As can be appreciated, modifications to this medical procedure can be used and/or other medical procedures can be used.

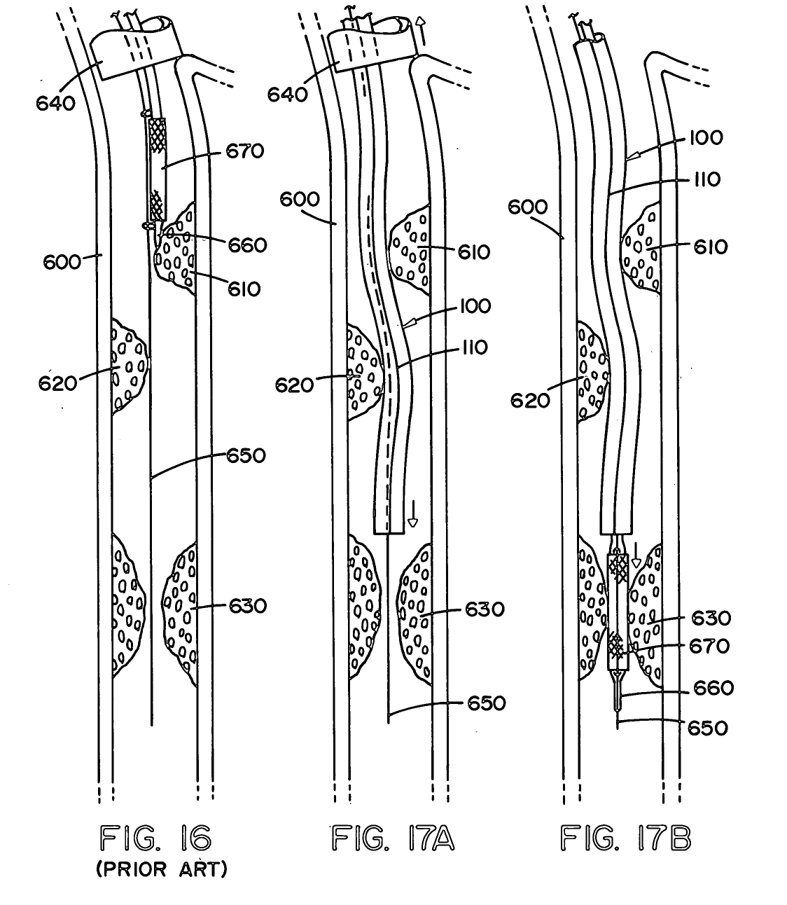

Referring now to FIG. 16, there is illustrated a blood vessel 600 that includes two minor deposits 610 and 620 and a blockage 630. Deposits 610 and 620 occlude less than 50% of the blood vessel, thus are typically not treated. Blockage 630 occludes over 50% of the blood vessel, thus is commonly treated by a stent. The guide catheter 640 is fed to a treatment area at the ostium of the blood vessel. A guide wire 650 is then fed through blockage 630. Due to the location and/or hardness of deposits 610 and 620, the angioplasty balloon 660 and stent 670 can not pass deposits 610 and 620, or cannot pass without damage to the stent as illustrated in FIG. 16. As such, in prior medical procedures, the angioplasty balloon and the stent had to be retracted back though the guide catheter and a cutting device, not shown, was then used to cut back some of the regions of deposits 610 and 620 so that the angioplasty balloon and stent could be fed to blockage 630. After the deposits were cut back, the guide catheter was then repositioned to the treatment area, an the guide wire was again fed to blockage 630. Thereafter, the angioplasty balloon and stent was fed to blockage 630 to repair the blockage. The delay in treatment of blockage 630 could be significant due to the cutting process. The cutting process could also be complicated and could cause potential damage to the blood vessel.

Referring now to FIGS. 17A and 17B, medical device 100 can be used to feed the angioplasty balloon and stent past deposits 610 and 620. The medical device can be positioned about the guide wire and angioplasty balloon at the back end of the guide catheter by slipping the guide wire and angioplasty balloon in the inner passageway of the medical device via the slit 110. As can be appreciated, the angioplasty balloon and stent can be removed from the guide wire prior to inserting the medical device about the guide wire. The medical device can then be fed through the guide catheter and about deposits 610 and 620 as indicated by the arrow in FIG. 17A. The durable, yet flexible, material of the medical device enables the medical device to be manipulated about deposits 610 and 620. A guide hub can be used to facilitate in the manipulation of the medical device. Thereafter, the angioplasty balloon and stent can be fed through the medical device and to the blockage 630 as illustrated by the arrow in FIG. 17B. Alternatively, the medical device can be fed about the stent, and then the medical device can fed about deposits 610 and 620 so as to enable the stent to be fed to blockage 630. The medical device can be used to facilitate in feeding the stent about deposits 610 and 620 thereby reducing the procedure time and/or protecting the stent and/or blood vessel from damage. As can be appreciated, modifications to this medical procedure can be used and/or other medical procedures can be used.

It will thus be seen that the objects set forth above, among those made apparent from the preceding description, are efficiently attained, and since certain changes may be made in the constructions set forth without departing from the spirit and scope of the invention, it is intended that all matter contained in the above description and shown in the accompanying drawings shall be interpreted as illustrative and not in a limiting sense. The invention has been described with reference to preferred and alternate embodiments. Modifications and alterations will become apparent to those skilled in the art upon reading and understanding the detailed discussion of the invention provided herein. This invention is intended to include all such modifications and alterations insofar as they come within the scope of the present invention. It is also to be understood that the following claims are intended to cover all of the generic and specific features of the invention herein described and all statements of the scope of the invention, which, as a matter of language, might be said to fall therebetween.

We claim:

1. A medical device designed to be used with a guide catheter, said medical device including a body having a cross-sectional area that is less than a cross-sectional area of an inner passageway of the guide catheter, said body including an internal passageway having a cross-sectional area that is adapted to be large enough to enable a treatment device to at least partially move within said internal passageway, said body having a longitudinal length that is at least about 10% of a longitudinal length of the guide catheter, said body at least partially formed of a flexible material.

2. The medical device as defined in claim 1, wherein said cross-section shape of said body is generally tubular.

3. The medical device as defined in claim 1, where said body has a longitudinal length that is greater than a longitudinal length of the guide catheter.

4. The medical device as defined in claim 2, where said body has a longitudinal length that is greater than a longitudinal length of the guide catheter.

5. The medical device as defined in claim 1, wherein said body includes at least one slit along a longitudinal length of said body.

6. The medical device as defined in claim 4, wherein said body includes at least one slit along a longitudinal length of said body.

7. The medical device as defined in claim 5, wherein said at least one slit is at least about 1% of said longitudinal length of said body.

8. The medical device as defined in claim 7, wherein said at least one slit is at least about 90% of said longitudinal length of said body.

9. The medical device as defined in claim 6, wherein said at least one slit is at least about 90% of said longitudinal length of said body.

10. The medical device as defined in claim 1, wherein said treatment device includes a device selected from the group consisting of a guide wire, an angioplasty balloon, a stent or combinations thereof.

11. The medical device as defined in claim 9, wherein said treatment device includes a device selected from the group consisting of a guide wire, an angioplasty balloon, a stent or combinations thereof.

12. The medical device as defined in claim 1, wherein said body includes at least one location marker.

13. The medical device as defined in claim 11, wherein said body includes at least one location marker.

14. The medical device as defined in claim 1, wherein at least a portion of said body includes at least one coating to reduce the roughness or surface friction of said coated body portion.

15. The medical device as defined in claim 11, wherein at least a portion of said body includes at least one coating to reduce the roughness or surface friction of said coated body portion.

16. The medical device as defined in claim 1, wherein at least a portion of said body includes at least one coating that includes at least one biological agent.

17. The medical device as defined in claim 11, wherein at least a portion of said body includes at least one coating that includes at least one biological agent.

18. The medical device as defined in claim 1, wherein at least a portion of said body includes at least one reinforcing, stiffening and/or strengthening components.

19. The medical device as defined in claim 11, wherein at least a portion of said body includes at least one reinforcing, stiffening and/or strengthening components.

20. The medical device as defined in claim 1, wherein said body includes one or more perforations.

21. The medical device as defined in claim 11, wherein said body includes one or more perforations.

22. The medical device as defined in claim 1, wherein said body includes a gripping surface at at least one proximal end of said body.

23. The medical device as defined in claim 11, wherein said body includes a gripping surface at at least one proximal end of said body.

24. The medical device as defined in claims 22, wherein said gripping surface includes a guide hub that is releasably connected to at least one proximal end of said body.

25. The medical device as defined in claims 23, wherein said gripping surface includes a guide hub that is releasably connected to at least one proximal end of said body.

26. A medical device designed to be used with a guide catheter, said medical device including a body having a cross-sectional area that is at least 5% less than a cross-sectional area of an inner passageway of the guide catheter, a majority of said body having a generally circular cross-sectional shape, said body including an internal passageway having a cross-sectional area that is adapted to be large enough to enable a treatment device to at least partially move within said internal passageway, a majority of said inner passageway of said body having a generally circular cross-sectional shape, said body having a longitudinal length that is at least about 10 cm and that is at least about 10% longer than a longitudinal length of the guide catheter, said body at least partially formed of a flexible polymer material, said treatment device includes a device selected from the group consisting of a guide wire, an angioplasty balloon, a stent or combinations thereof.

27. The medical device as defined in claim 26, wherein said body includes at least one slit along at least about 50% of a longitudinal length of said body.

28. The medical device as defined in claim 26, wherein said body includes at least one location marker.

29. The medical device as defined in claim 26, wherein at least a portion of said body includes at least one coating to reduce the roughness or surface friction of said coated body portion.

30. The medical device as defined in claim 26, wherein at least a portion of said body includes at least one coating that includes at least one biological agent.

31. The medical device as defined in claim 26, wherein at least a portion of said body includes at least one reinforcing, stiffening and/or strengthening components.

32. The medical device as defined in claim 26, wherein said body includes one or more perforations.

33. The medical device as defined in claim 26, wherein said body includes a gripping surface at at least one proximal end of said body, said gripping surface includes a guide hub that is releasably connected to said at least one proximal end of said body.

34. A method of treating a diseased area of a body passageway comprising:
a. inserting a guide catheter at least partially through a body passageway until one end of said guide catheter is within about 18 inches or less from the diseased area, said guide catheter having an inner passageway; and,
b. inserting a medical device into at least a majority longitudinal length of said inner passageway of said guide catheter, said medical device including a body having a cross-sectional area that is at least 5% less than a cross-sectional area of said inner passageway of the guide catheter, said body including an internal passageway having a cross-sectional area that is adapted to be large enough to enable a treatment device to at least partially move within said internal passageway, said body having a longitudinal length that is at least about 10 cm.

35. The method as defined in claim 34, including the step of moving a treatment device at least partially through said internal passageway of said body of said medical device when said medical device is at least partially positioned in said inner passageway of said guide catheter, said treatment device including a device selected from the group consisting of a guide wire, an angioplasty balloon, a stent or combinations thereof.

36. The method as defined in claim 34, wherein said medical device has a longitudinal length that is greater than a longitudinal length of said guide catheter and including the step of inserting said medical device in said inner passageway of said guide catheter until as one end of said medical device extends from a first end of said guide catheter and another end of said medical device extends from a second end of said guide catheter.

37. The method as defined in claim 34, wherein said body of said medical device includes at least one slit along at least about 50% of a longitudinal length of said body.

38. The method as defined in claim 34, wherein said body of said medical device includes at least one location marker.

39. The method as defined in claim 34, wherein at least a portion of said body of said medical device includes at least one coating to reduce the roughness or surface friction of said coated body portion.

40. The method as defined in claim 34, wherein at least a portion of said body of said medical device includes at least one coating that includes at least one biological agent.

41. The method as defined in claim 34, wherein at least a portion of said body of said medical device includes at least one reinforcing, stiffening and/or strengthening components.

42. The method as defined in claim 34, wherein said body of said medical device includes one or more perforations.

43. The method as defined in claim 34, wherein said body of said medical device includes a gripping surface at at least one proximal end of said body, said gripping surface includes a guide hub that is releasably connected to at least one proximal end of said body.

44. The method as defined in claim 34, including the step of directing an end of said medical device to close proximity to a portion of an angioplasty balloon that has at least partially adhered to another treatment device and withdrawing said balloon until said balloon releases from said treatment device.

45. The method as defined in claim 34, including the steps of moving a portion of said medical device to a treatment site, at least partially withdrawing an end of said guide catheter from said treatment site, and subsequently sliding said guide catheter on said medical device until the end of said guide catheter is again positioned at or closely to the treatment site.

46. The method as defined in claim 34, including the steps of positioning on end of said medical device at or closely to said diseased area in said body passageway and then moving at least one of said treatment devices at least partially through said internal passageway of said body of said medical device to at least partially protect said treatment device from said body passageway until said treatment device is positioned at or closely to said diseased area.

47. The method as defined in claim 34, including the steps of positioning on end of said medical device at or closely to at least of said treatment devices and then at least partially drawing said treatment device into and through said internal passageway of said body of said medical device so as to at least partially protect an inner surface of the body passageway.
